(19) United States
(12) Patent Application Publication
(10) Pub. No.: US 2021/0331153 A1
(43) Pub. Date: Oct. 28, 2021

(54) METHOD FOR APPLYING, ON A SOLID SUPPORT, AT LEAST ONE BINDING PARTNER TO A MOLECULE

(71) Applicant: BIOMÉRIEUX, Marcy l'Etoile (FR)

(72) Inventors: Patrick BLASCO, Vaugneray (FR); Perrine COSIN, Thurins (FR)

(73) Assignee: BIOMÉRIEUX, Marcy l'Etoile (FR)

(21) Appl. No.: 16/625,346

(22) PCT Filed: Jun. 19, 2018
(86) PCT No.: PCT/FR2018/051464

§ 371 Date: Dec. 20, 2019

(30) Foreign Application Priority Data
Jun. 20, 2017 (FR) 1755602

(51) Int. Cl. B01L 3/02; B01L 9/00; G01N 33/543

(52) U.S. Cl. CPC B01L 3/0275; B01L 9/54; B01L 2300/069; B01L 2300/0832; G01N 33/54366

(57) ABSTRACT

A process for applying, in a tubular solid support, at least one binding partner P1 to an analyte to be detected or quantified in a test sample, including steps: (i) connect the support to a suction-discharge device, (ii) draw up into the support, by one of its ends, a solution including the binding partner P1, called sensitization solution S1, contained in a container, called container C1, (iii) continue contact between solution S1 and the inner surface of the support for a time between 0 s and 11 min, (iv) discharge the solution S1 into a container, which is optionally the container C1, steps (ii) to (iv) forming a cycle that can be repeated at least once, over a total duration of at least 1 min and at most 2.5 h. Also, the use of the coated support for the detection or quantification of an analyte in a test sample.

The present invention relates to the field of in vitro detection of analytes in test samples likely to contain these analytes. In particular, the invention relates to a novel process for applying binding partners to the desired analyte on a tubular solid support, which support is then used for the detection of the analyte.

In the field of analysis of test samples likely to contain analytes of interest, it is known to use methods based on specific measurements such as signal measurements. The analysis of the test sample may include the use of a binding partner to the analyte to be detected or quantified in the sample. The binding partner makes it possible to isolate the analyte to be tested in order to implement its detection by obtaining a reaction product emitting a signal such as a colored signal or a fluorescence signal.

The binding partner used to isolate the analyte from the test sample is most often bound to the surface of a solid support. Different solid supports can be used such as solid supports, such as beads, hollow supports having a bottom, called closed hollow supports or hollow supports closed at one end, such as wells, or bottomless hollow supports, called open hollow supports or hollow supports open at each end, in the form of tubes, such as cones or pipettes.

When the supports are hollow, open or closed supports, the binding partners to the analyte used are fixed, for example by adsorption (it is then said that they are coated), inside the support, on its inner surface.

Usually, the application of binding partners to the inner surface of hollow supports is made by placing a solution containing the binding partners on the inner surface of the support in static form for a long period of more than 6 hours, and on average overnight, as described in patent application FR2417094. Because of this long preparation time, biological analysis using these media cannot be carried out quickly because it is necessary to wait at least the time for the preparation of the media and, in most cases, the preparation of the media and the analysis must be carried out over at least two days. Indeed, for a biological analysis to be acceptable, particularly in in vitro diagnostics, and especially in cases where an analysis report is urgent, it is necessary that the total analysis time be as short as possible.

As a result, as a matter of routine, the substrates are prepared in advance, dried and then stored in desiccant bags to avoid any stability problems. They are then used with other elements necessary for the analysis, such as reagents.

The application of the binding partners on the surface of the hollow supports is therefore done over a long period of time, generally using a single solution containing these binding partners, called the sensitization or coating solution, in a closed system, i.e. the same solution whose concentration of binding partners is always used when they are applied on the inner surface of the hollow supports. It is then necessary for the solution to contain these binding partners in large excess, which is costly. Nevertheless, the biggest disadvantage with this method is the contact time required to obtain the right amount of applied binding partner.

Another process for applying binding partners in closed hollow supports, such as microplates, has been developed. It consists of bringing together a sensitization solution containing the binding partners, then possibly shaking the plate by means of a vibration or orbital motion shaking plate for several hours, on average 4 hours. Even if the coating time is a little shorter than a static process, it is far from acceptable. In addition, again, since shaking takes place in a closed system, as with static application, it is necessary that the sensitization solution contains the binding partners in large excess. Another disadvantage of this process is that it cannot be used with open hollow supports. Finally, the disadvantage of this process is that the supports thus coated by the binding partners are not very reproducible because the process cannot be automated.

Compared to hollow supports closed at one end, the advantage of hollow supports open at each end is that they can also be used for pipetting and transporting reaction liquids, including the sample. They are used in particular to draw up liquids and then discharge them.

An example of a system using such an open hollow support in which the binding partners are applied is the VIDAS® system (bioMérieux). This instrument uses a pipette tip+test strip pair for the analysis of test samples. The test strip, introduced into the system, consists of several cuvettes, some of which are filled with predetermined liquids, useful for analysis. The test strip also includes a cuvette adapted to receive a sample to be tested and a cuvette for reading the test result. Thus, in this system, the pipette tip contains part of the reagents necessary for the emission of the signal and the test strip contains the other part of the reagents. When analyzing this test sample, the pipette tip is used to draw a quantity of said sample and deposit said sample inside the different cuvettes present in the test strip. The liquids present inside the different cells can react with the sample to obtain, at the end of a liquid transfer cycle, for example from one cell to another, a liquid, or reaction medium, on which the measurements of a fluorescence signal are made. If the analyte to be detected or quantified is present in the test sample, it will bind to the binding partner present on the surface of the pipette tip, and then the liquids present in the different cuvettes will be used to detect this analyte. With the VIDAS® instrument, the binding partners are applied in the pipette tips beforehand for about 12 hours, statically, so that the kits sold contain pre-coated pipette tips and are stored for later use.

Regardless of how the binding partner is applied and regardless of the medium, this system requires the use of at least two instruments of different nature and functionality, one to apply the capture partners and the other to perform the analysis.

The present invention therefore aims to overcome the disadvantages of the state of the art by proposing a process that dynamically and rapidly applies one or more binding partners to the inner surface of an open hollow support, thus significantly shortening the application time of the binding partners. This way, it is possible to immediately continue the analysis, thus avoiding the storage of the coated substrate. Moreover, with such a process, a single type of instrument may be sufficient for both the application of the binding partner(s) and the analysis, even if these two steps are not implemented immediately one after the other. Finally, having such a process, which significantly shortens the application time of the binding partner(s) and uses a sensitization solution in which the number of binding partners is reduced, also allows for significant economic savings when producing open hollow supports.

To achieve such an objective, the subject matter of the invention relates to a process for applying, in a tubular solid support, optionally flared, having a circular or ellipsoidal opening at each end, such as a pipette tip or a pipette, at least one binding partner P1 to an analyte to be detected or quantified in a test sample, comprising the following steps:

(i) connect the solid support to a suction-discharge device,
(ii) draw up into the support, by one of its ends, a solution comprising said at least one binding partner P1, called sensitization solution S1, contained in a container, called “container C1”,
(iii) continue contact between the sensitization solution S1 and the inner surface of the solid support for a time between 0 s and 11 min,
(iv) discharge the sensitization solution P1 into a container, which is optionally said container C1,

steps (ii) to (iv) forming a cycle that can be repeated at least once, over a total duration of at least 1 min and at most continue contact between 2.5 h.

The process according to the invention has in combination one and/or other of the following features:

the contact time of step (iii) between the sensitization solution S1 and the inner surface of the solid support is between 2 s and 1 min, preferably between 5 s and 25 s, more preferably between 6 s and 20 s;

steps (ii) to (iv) are repeated 10 to 150 times, preferably 35 to 80 times;

the total of the repeated cycles is between 10 and 20 min and is preferably 15 min;

the following steps (v) to (vii) are implemented when the suction (ii)/contact (iii)/discharge (iv) cycles are completed:

(v) draw up into the support in which the at least one binding partner P1 is applied, a wash solution W1 contained in a container called container CW1,
(vi) continue contact between the wash solution W1 and the inner surface of the solid support for a time between 0 s and 5 min,
(vii) discharge the wash solution W1 into a container, which may or may not be said container CW1,

steps (v) to (vii) forming a cycle that can be repeated at least once, over a total duration of at least 1 min and at most 2.5 h;

the sensitization solution S1 also contains at least one binding partner P2 to the binding partner P1 to the analyte.

The binding partner P2 to the binding partner P1 to the analyte to be detected or quantified can advantageously be applied to the inner surface of the solid hollow support according to the same process of the invention as described above by repeating the following steps:

(a) connect the solid support to a suction-discharge device,
(b) draw up into the solid support, by one of its ends, a solution comprising said at least one binding partner P2, called sensitization solution S2, contained in a container, called container C2,
(c) continue contact between the sensitization solution S2 and the inner surface of the solid support for a time between 0 s and 11 min,
(d) discharge the sensitization solution S2 into a container, which is optionally said container C2,
steps (b) to (d) forming a cycle that can be repeated at least once, over a total duration of at least 1 min and at most 2.5 h.

The process for applying the partner P2 has in combination one and/or more of the following features:

it also includes the following steps (e) to (g), implemented when the suction (b)/contact (c)/discharge (d) cycles are completed:

(e) draw up into the support in which the at least one binding partner P2 is applied, a wash solution W2 contained in a container called container CW2,

(f) continue contact between the wash solution W2 and the inner surface of the solid substrate for a time of between 0 s and 5 min,

(g) discharge the wash solution W2 into a container, which is optionally said container CW2,

steps (e) to (g) forming a cycle that may be at least once, over a total duration of at least 1 min and at most 2.5 h;

it also includes the application, in said solid support, after the suction (b)/contact (c)/discharge (d) cycles, of said at least one binding partner P1;

the application of said at least one binding partner P1, when implemented, is carried out according to an application process of the invention, as described above.

The open hollow solid support, coated with the analyte binding partner P1, optionally via the binding partner P1 to P2, is advantageously used for the detection or quantification of the analyte contained in a test sample. Also, another subject matter of the invention relates to a process for the in vitro detection or quantification of an analyte in a test sample likely to contain said analyte, said process using at least one tubular solid support, optionally flared, having a circular or ellipsoidal opening at each end, such as a pipette tip or a pipette, in which at least one binding partner P1 to an analyte to be detected or quantified in the test sample is applied according to the application process of the invention, which process of detection or quantification comprises the steps of contacting the test sample with the solid support and detecting the binding, if the analyte is present, of said analyte and said at least one binding partner P1.

The detection or quantification process has in combination one or more of the following features:
the binding partner P1 is an immunoassay partner;
the detection of the binding of said analyte is carried out by a sandwich test using another binding partner to the analyte, optionally of a different nature, which is labelled, or by a competitive test using a labelled compound competing with the analyte to be detected or quantified;
the detection of the analyte is carried out using an enzyme and an enzyme substrate catalyzed by said enzyme, preferably alkaline phosphatase and 4-methylumbelliferyl phosphate;
the steps of contacting the test sample with the solid support and detecting the binding, if the analyte is present, of said analyte and said at least one binding partner P1 are carried out immediately after the process of applying at least one binding partner P1 in said solid support as defined above, preferably with the same instrument or an instrument of the same range;
the steps of contacting the test sample with the solid support on which said at least one binding partner P1 is applied, and detecting the binding of said analyte and said at least one binding partner P1, include the following steps consisting in:
(1) drawing up into said solid support, by one of its ends, said sample contained in a container, called container CE, leaving in contact and discharging said sample into a container, which is optionally said container CE,
(2) drawing up into said solid support, at the same end, a solution comprising a compound conjugated to a label, such as another binding partner to the analyte or a compound competing with the analyte, called conjugate solution CS, said conjugate solution CS being contained in a container called container CC, allowing said conjugate solution CS to contact and discharge into a container, which is optionally said container CC,
(3) if the label uses a detection substrate, drawing up into said solid support, at the same end, a solution comprising a detection substrate with which the label will react, called substrate solution SS, said substrate solution SS being contained in a container called container CS, leaving in contact and discharging said substrate solution into a container, which is optionally said container CS, and
(4) measuring the transmitted signal.

Of course, the process can include one or more washing steps between steps (1) and (2) and steps (2) and (3), implemented as described below.

Application in an open hollow solid support of at least one binding partner means that the at least one binding partner is fixed inside the support by any means known to the skilled person such as adsorption, covalent binding, hydrogen binding, electrostatic binding and ionic binding, etc. The type of fixing of the binding partners inside the hollow support depends on the nature of the support and in particular its surface. Thus, the support can be made of plastic, such as polycarbonate, styrene-butadiene copolymers, polymers (polystyrene, polypropene, polypropylene, polyethylene, etc.) or stainless steel, with or without pretreatment of the inner surface. The inner surface of the hollow support may have been functionalized by reactive groups that will result in the formation of a covalent bond with the binding partner. The inner surface of the hollow support may also have undergone treatments to improve the fixation of the binding partners, such as ionizing radiation treatment such as irradiation with X-rays, accelerated electrons or gamma radiation. Such pretreated or untreated supports are commercially available, such as from Thermofisher.

Analyte to be detected or quantified means any molecule representative of the presence of microorganisms or a disease to be detected, characterized or monitored. It also includes the screening of drugs or physical-active substances, drugs and the follow-up of treatments in patients. Preferably, the detection or quantification of this analyte is performed by an immunoassay which is a test widely known to the skilled person, involving immunological reactions between this analyte to be detected and a binding partner(s) to this analyte. Therefore, examples of analytes include antibodies, for example autoantibodies or antibodies against pathogen proteins, for example viruses such as HIV, SIV, FIV, HCV, HBV, HAV, HEV, VZV, CMV, EBV, HSV1, HSV2, bacteria such as Mycobacterium tuberculosis, Mycobacterium bovis, Mycobacterium leprae, Borrelia burgdorferi strict sensu, Borrelia afzelii, Borrelia garinii, Borrelia spielmanii, Clostridium difficile, Clostridium botulinum, salmonella, Klebsiella, Legionella, Proteus, Klebsiella, Escherichia coli, Shigella, Pseudomonas aeruginosa, Staphylococcus aureus, Treponema pallidum, yeasts such as Candida albicans, fungi such as Aspergillus fumigatus, Mucorales, etc.), proteins different from antibodies, for example biomarkers such as procalcitonin (PCT), thyroid hormones (for example TSH), cardiac troponins (TnI, TnT, and associated complexes), BNP, NT-proBNP, D-dimer, CRP, S100B, L-FABP, H-FABP, cancer markers such as CA-15-3, CA-19-9, etc., or pathogen proteins, peptides, for example pathogen protein fragments as described above, and haptens, for example steroids or vitamins.

The analyte is likely to be contained in a test sample that may be of various origins, such as food, environmental, biological, veterinary, clinical, pharmaceutical or cosmetic origin.

Food samples include, but are not limited to, a sample of milk products (yogurts, cheeses, etc.), meat, fish, eggs, fruit, vegetables, water, beverages (milk, fruit juices, soda, etc.). Of course, these food samples may also come from more elaborate sauces or dishes or from unprocessed or partially processed raw materials. A food sample may also be obtained from animal feed, such as oilcake or animal meal. All these samples, if they are not liquid, are preprocessed to be in liquid form.

As indicated above, the sample may be of environmental origin and may consist, for example, of a surface sample, water, air, etc.

The sample may also consist of a biological sample, of human or animal origin, which may consist of samples of biological fluid (urine, whole blood or derivatives such as serum or plasma, saliva, pus, cerebrospinal fluid, etc.), stool (for example cholera diarrhea), nose, throat, skin, wounds, organs, tissues or isolated cells, swab samples. This list is obviously not exhaustive.

In general, the term “sample” refers to a part or quantity, more particularly a small part or quantity, taken from one or more entities for analysis. This sample may have undergone prior treatment, involving for example mixing, dilution or grinding steps, particularly if the starting entity is in a solid state.

Binding partner P1 to the analyte to be detected or quantified in a test sample means any partner capable of binding to said analyte. Of course, the concepts of analyte and binding partner are quite distinct from each other. The analyte is the compound present in the test sample, while the binding partner P1, which is applied according to the process of the invention, is the compound that will bind to the analyte. The nature of the binding partner P1 depends on the nature of the analyte and the type of test used. Preferably, the test will be an immunoassay.

The term “immuno” in “immunoassay” for example, is not to be considered in this application as strictly indicating that the binding partner is an immunological partner, such as an antibody. Indeed, the skilled person also uses this term widely when the binding partner, also called ligand, is not an immunological partner but is for example a receptor for the analyte to be measured. Thus, it is known to speak of the enzyme-linked immunosorbent assay (ELISA) for assays that use non-immunological binding partners, more broadly called ligand binding assays, which could be restated as assays using ligand binding, whereas the term “immuno” is included in the acronym ELISA. For the sake of clarity, the Applicant will use the term “immuno” throughout the application for any test or assay using a binding partner, even when it is not an immunological partner.

Examples of immunoassay binding partners P1 include binding partners of an immunological nature or origin such as antibodies (monoclonal or polyclonal) and antibody fragments (such as Fab, Fab′, F(ab′)2), scFv (single chain variable fragment), dsFv (double-stranded variable fragment), well known to the skilled person, as well as binding partners that are not of an immunological nature or origin such as proteins different from antibodies, peptides, oligonucleotides, nanofitins, analyte receptors if they exist, aptamers, DARPins or any other molecule that is known to have an interaction with said analyte.

Nanofitins (trade name) are small proteins that, like antibodies, are able to bind to a biological target, allowing it to be detected, captured or simply targeted within an organism.

Aptamers are oligonucleotides, usually RNA or DNA, identified in banks containing up to 1015 different sequences, by an in vitro combinatorial selection method called SELEX for “Systematic Evolution of Ligands by Exponential Enrichment” (Ellington A D and Szostak J W., 1990). Most aptamers are composed of RNAs, due to the ability of the RNA to adopt varied and complex structures, which allows the creation of cavities of various geometries on its surface, allowing various ligands to be attached. These are biochemical tools of interest that can be used in biotechnological, diagnostic or therapeutic applications. Their selectivity and ligand binding properties are comparable to those of antibodies.

Designed ankyrin repeat proteins (DARPins) (Boersma Y L and Plütckthun A, 2011) are another class of proteins that mimic antibodies and can bind with high affinity and selectivity to target proteins. They are derived from the family of ankyrin proteins, which are adaptive proteins that bind integral membrane proteins to the spectral/actin network that constitutes the “spine” of the plasma cell membrane. The structure of ankyrins is based on the repetition of a motif of about 33 amino acids and the same is true for DARPins. Each motif has a secondary structure of the helix-turn-helix type. DARPins contain at least three, preferably four to five repeated motifs and are obtained by screening combinatorial banks.

The sensitization solution S1 includes the binding partner P1 in a concentration that skilled persons will adapt according to the sensitivity of the test they wish to perform, the binding partner P1 to be applied in the solid support, the analyte sought and the nature of the sample. Thus, for example, when the sensitization solution includes an antibody or antibody fragment, it may be used in a concentration between 0.01 and 100 μg/mL. Unlike a conventional application process, in static or closed hollow support, the quantity of binding partner P1 can be lower, for example by 10% compared to the concentration used in static.

The sensitization solution S1 also includes all components necessary to facilitate the application of the binding partner P1 in the solid support, such as pH buffers, for example carbonate, phosphate, TRIS, HEPES buffers, salts such as NaCl, preservatives such as azide, MIT; stabilizers such as glycerol, Tween 20, SDS, and saturation or passivation proteins such as BSA, casein.

The container C1 containing the sensitization solution S1 can be any container that contains a solution containing biological compounds such as the binding partners P1. The only requirement is that the walls of the container are inert with respect to the binding partners P1 so that the latter do not attach themselves to the walls. Thus, the container C1 can be made of plastic, such as ethylene vinyl acetate (EVA copolymer), polyethylene, polypropylene copolymer, fluorinated ethylene propylene (Nalgene), glass, or stainless steel. It can be in any form, for example, vial, well, canister, box, jerry cans. This container C1 can be closed before its implementation in the process of the invention and then opened during the suction step. For example, this container C1 may be a well covered with an aluminum foil cover that will be pierced by the solid support itself when it draws up the sensitization solution.

Implemented with the same instrument means that the steps concerned use a single instrument, whereas implemented with two instruments of the same range means that the steps concerned use two different instruments but from the same manufacturer and with comparable features to be interchangeable. The latter case refers indifferently to instruments of the same range or of the same type. Examples include the Applicant's VIDAS® range, which includes the VIDAS®, mini VIDAS® and VIDAS® 3 instruments. Thus, for example, the application of the binding partner(s) can be implemented with a VIDAS® instrument, then the analysis can be implemented with another VIDAS® instrument or with a VIDAS® 3 instrument.

The first step in the process of the invention relates to the connection of the solid support to a suction-discharge device. The connection can be made manually or mechanically by the instrument in which the support and the suction-discharge device are located.

The process of the invention includes several suction (ii)/contact (iii)/discharge (iv) cycles. The first step in this cycle is the suction step (ii) which consists in inserting the sensitization solution S1 into the hollow solid support by one of its ends, without overflowing by the other end. It may be implemented by any method known to the skilled person. For example, this suction step can be carried out by making a pressure difference in the solid support, in particular by using a piston driven by any means known to the skilled person, such as a screw coupled to a motor, forming a pump unit or by using a vacuum creation device. The pump unit can be at a varying distance from the solid support, for example through a tubing. Seals between the solid support and the pump unit are present to seal it. All these elements are an example of a suction/discharge device. The skilled person will adapt the suction rate and power of the pump according to the amount of sensitization solution to be introduced into the solid support and, if necessary, according to the length and cross-section of the tubing between the pump and the solid support. Thus, for example, in the case of the VIDAS® instrument where the pump unit is close to the pipette tip, the suction speed will be between 10 and 180 μL/second. As indicated above, the solid support can be used to directly draw up the sensitization solution from the container C1 or the suction can be carried out via a tubing on the one hand connected to the end of the solid support by which the sensitization solution P1 is drawn up and on the other hand immersed in the container C1.

Unlike the suction step (ii), the discharge step (iv) consists in removing the sensitization solution S1 from the hollow solid support by one of its ends. The end through which the sensitization solution is pushed back can be the same as the one used to aspirate, as for example in the case of the VIDAS® instrument, or it can be the opposite end.

The discharge container that collects the discharged sensitization solution can be the container C1, for example when the same end of the solid support is used for suction and discharge. The discharge container may also consist of a waste bin.

The discharge step can be carried out by any method known to the skilled person, as described for the suction step. Conveniently, the same system, for example a pump unit as described above, is used for both suction and discharge steps.

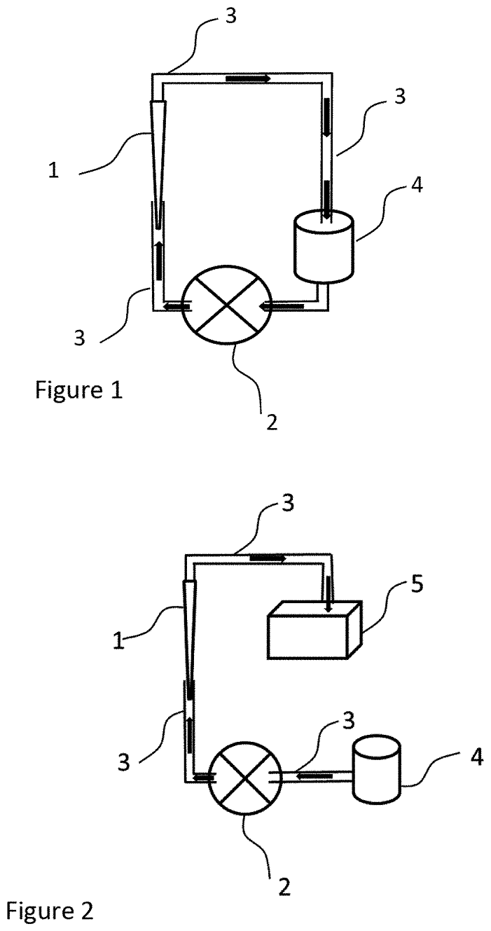

Various other features emerge from the description given below with reference to the appended FIGS. 1 to 8, which show, by way of non-limiting examples, embodiments of the subject matters of the invention.

FIG. 1 shows a diagram for the application of a binding partner, in a solid pipette tip-type support, with a loop coating system.

FIG. 2 shows a diagram for the application of a binding partner, in a solid pipette tip-type support, with an in-line system.

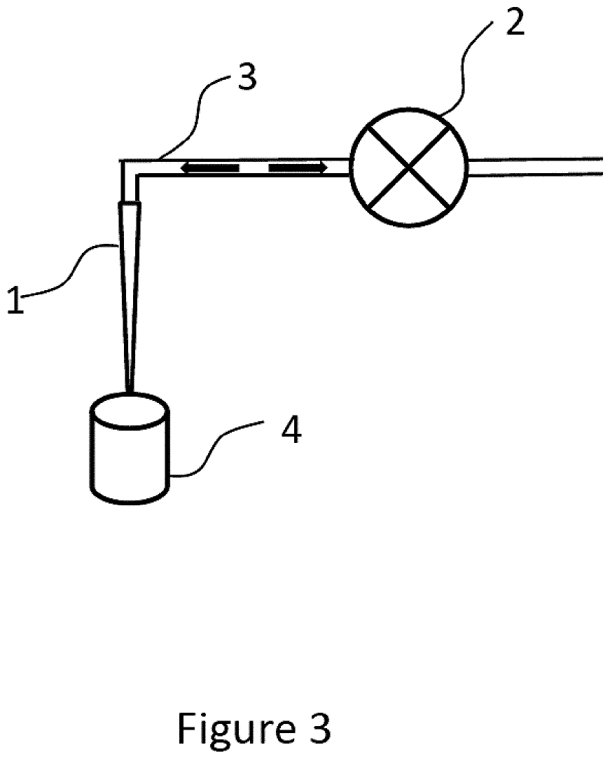

FIG. 3 shows a diagram for the application of a binding partner, in a solid pipette tip-type support, with a reciprocating system, as implemented for example in the VIDAS® instrument.

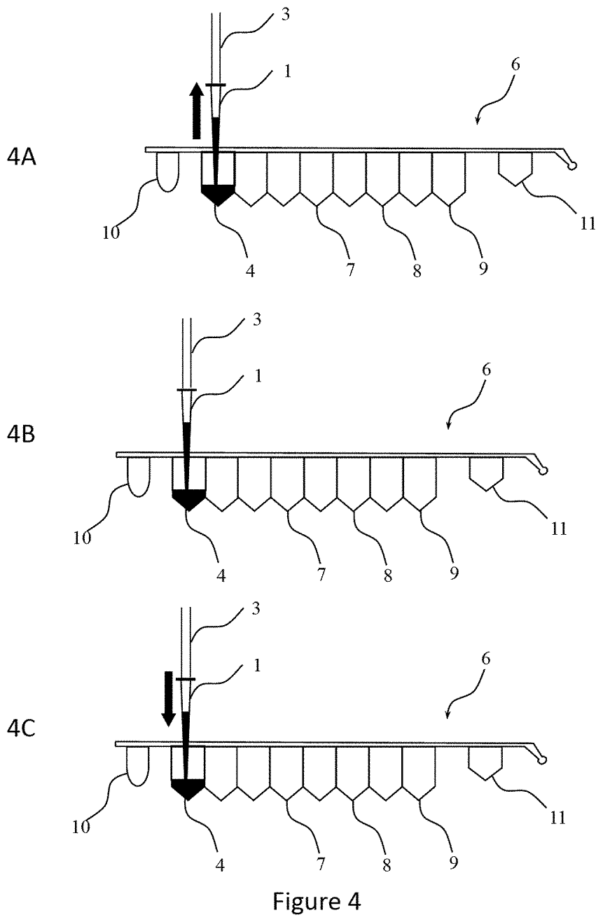

FIG. 4 shows a strip and a VIDAS® pipette tip when implementing a suction cycle (FIG. 4A)/contact (FIG. 4B)/discharge (FIG. 4C) according to an embodiment of the invention for the application of a binding partner, the strip being made up of several containers including the container S1 or S2.

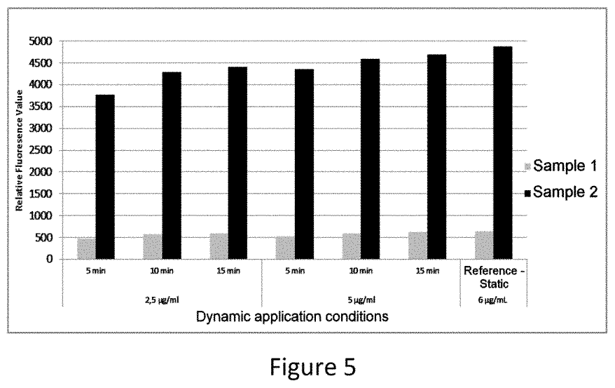

FIG. 5 is a histogram-type representation of the relative fluorescence values (RFVs) for both sample types 1 and 2 when detecting TSH with the VIDAS® instrument, as a function of the different application conditions of the binding partner (dynamically with a concentration of binding partner and a contact time that varies—2.5 μg/mL for 5, 10 or 15 min or 5 μg/mL for 5, 10 or 15 min, or static with a concentration at 6 μg/mL for 20 h).

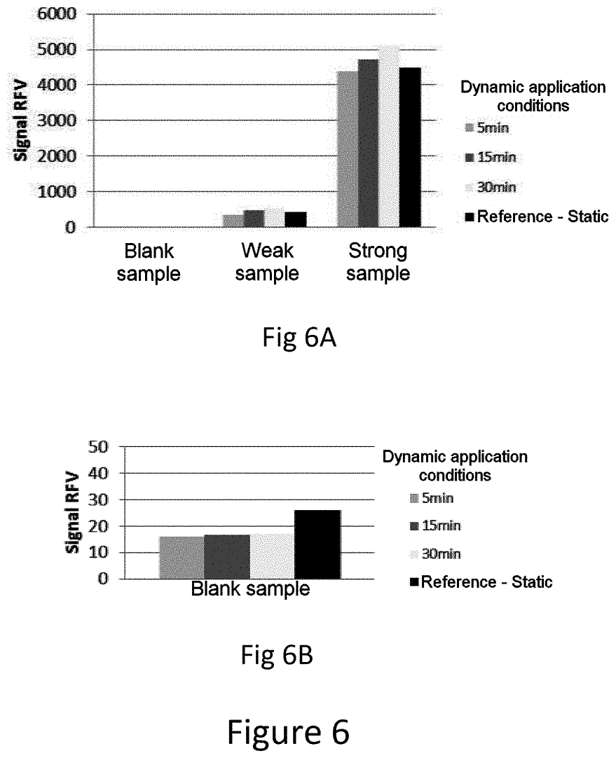

FIG. 6 is a histogram-type representation of the relative fluorescence values (RFVs) for the three sample types (blank, low and high—FIG. 6A) or only for the blank sample (representation of the histograms in FIG. 6A with enlarged scale—FIG. 6B) when detecting anti-Tg antibodies with the VIDAS® instrument, as a function of the different application conditions of the binding partner (dynamic for 5, 15 or 30 min, or static for 20 h).

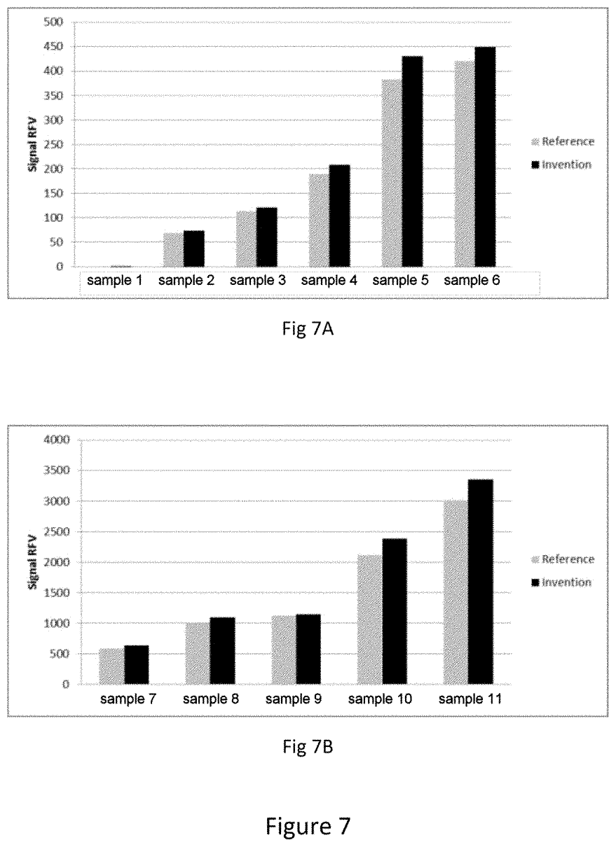

FIG. 7 is a graph showing the RFV signal as a function of TnI concentration when detecting TnI with the VIDAS instrument (FIG. 7A: low TnI concentration and FIG. 7B: high TnI concentration) using a pipette tip coated with 3 binding partners according to the process of the invention (invention) or according to the prior art (reference), namely biotinylated BSA, then streptavidin, then a mixture of biotinylated anti-TnI antibodies.

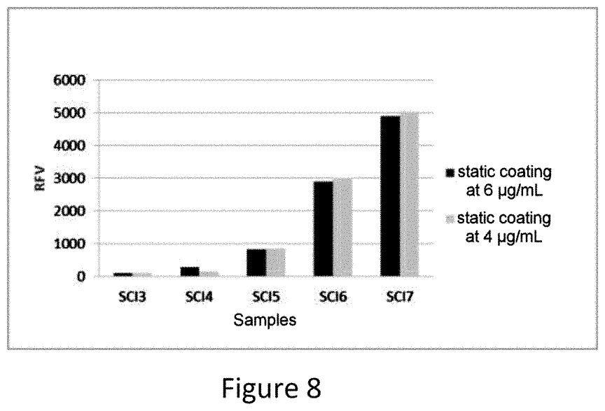

FIG. 8 is a histogram giving the RFV signal for five samples with increasing TSH concentration using either a pipette tip coated with an anti-TSH antibody according to the invention (dynamic coating at 4 μg/mL) or a pipette tip coated with an anti-TSH antibody according to the prior art (static coating at 6 μg/mL).

FIGS. 1 to 3 are particular embodiments of implementation of suction/contact/discharge cycles according to the process of the invention.

According to FIG. 1, the device for applying the binding partner P1 in an open hollow support 1, of pipette tip type, comprises a container C1 4 containing the sensitization solution S1 containing said binding partner P1, a pump 2 which allows the sensitization solution to circulate, via a tubing system 3, in the indicated arrow direction. The first step consists of drawing up, in the direction of the arrow, using pump 2, the sensitization solution from the container C1 4 into the support 1, then optionally leaving the sensitization solution and the support 1 in contact, then discharging, still in the direction of the arrows, using the pump 2, the sensitization solution from the support 1 into the container 4, these steps being repeated. Thus, the sensitization solution is taken up at each step into the container 4, enters through one of the two ends of the support 1 and is discharged into the container 4 through the other end of the support 1.

According to a variant of this embodiment and as indicated above, the sensitization solution S1, after being drawn up into the support 1 by one of its ends, is discharged into the container C1 4 by the same end.

According to FIG. 2, the device for applying the binding partner P1 in an open hollow support 1, of pipette tip type, comprises a container C1 4 containing the sensitization solution S1 containing said binding partner P1, a pump 2 which allows the sensitization solution to circulate, via a tubing system 3, in the indicated arrow direction. The first step consists of drawing up, in the direction of the arrow, using the pump 2, the sensitization solution from the container C1 4 into the support 1, then optionally leaving the sensitization solution and the support 1 in contact, then discharging, still in the direction of the arrows, using the pump 2, the sensitization solution from the support 1 into the container 5 which is a waste container, these steps being repeated and the sensitization solution being taken up at each step into the container C1 4. Thus, the sensitization solution is taken up at each step into the container 4, enters through one of the two ends of the support 1 and is discharged into the container 5 through the other end of the support 1.

According to a variant of this embodiment, the suction/contact/discharge cycles are carried out using the same end of the support 1 and it is only during the last cycle that the sensitization solution is discharged into a waste container.

According to FIG. 3, the device for applying the binding partner P1 in an open hollow support 1, of pipette tip type, comprises a container C1 4 containing the sensitization solution S1 containing said binding partner P1, a pump 2 which allows the sensitization solution to circulate, via a tubing system 3, in the indicated arrow direction. The first step consists of drawing up, in the direction of the arrow going to the right, using the pump 2, the sensitization solution from the container C1 4 into the support 1, then optionally leaving the sensitization solution and support 1 in contact, then discharging, still in the direction of the arrow to the left, using the pump 2, the sensitization solution from the support 1 into the container 4, these steps being repeated. Thus, the sensitization solution is taken up at each step into the container 4, enters through one of the two ends of the support 1 and is discharged into the container 4 from the same end.

An example of a process consisting of drawing up and discharging the sensitization solution using the same end of the hollow support is shown in FIG. 4 which represents a strip 6 and a VIDAS® pipette tip 1. The test strip 6 contains different wells, including a well 10 to receive the test sample, a well 4 containing the sensitization solution S1, a reading well 11 in which the fluorescence will be read after the biological analysis of the sample is carried out, and wells 7, 8, 9 which may contain different elements such as wash buffers or a waste well in which the fraction of sensitization solution that comes into contact with the pipette tip is discharged. FIG. 4A shows the first step of the process of the invention, consisting in drawing up into the pipette tip 1, via a tubing 3 connected to a suction-discharge device (not shown in FIG. 4, but as shown in FIG. 3), a fraction of the sensitization solution contained in the well 4. FIG. 4B shows the second step during which the sensitization solution and the pipette tip 1 are left in contact. Finally, FIG. 4C shows the discharge step consisting of discharging the sensitization solution into the well 4.

The contact step (iii) between the sensitization solution S1 and the solid support is performed for a time between 0 s and 11 min. When the contact time is 0 s, it means that the suction and discharge steps follow one another, without any pause time between the two. The contact time can also be at most 10 min, 9 min, 8 min, 7 min, 6 min, 5 min, 4 min, 3 min, 2 min, 1 min, 55 s, 50 s, 45 s, 40 s, 35 s, 30 s, 25 s, 20 s or 15 s. The contact time can be at least 1 s, 2 s, 3 s, 4 s, 5 s, 6 s, 7 s, 8 s, 9 s, 10 s, 11 s, 12 s, 13 s, 14 s, 15 s, 20 s, 30 s, 35 s or 40 s.

The contact time also varies according to the number of cycles implemented, which is at least two. The minimum and maximum number of cycles depends on the total duration of the application process, depending on the contact time chosen. Thus, the number of repetition cycles of steps (i) to (ii) may be at least 2, 3, 4, 5, 6, 7, 8, 9, 10, 11, 12, 13, 14, 15, 16, 18, 20 or 25 and at most 30, 35, 40, 45, 50, 55, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 139, 150, 185, 200, 232, 250, 278, 300, 325, 371, 400, 417, 464, 500, 510, 556, 600, 603, 649, 700, 800, 900, 1000, 2000, 2500, 3000, 4000, 5000 or 6000 times.

The total time of the process is at least 1 min and at most about 2.5 h. This total time can be at least 2, 3, 4, 5, 6, 7, 8, 9, 10, 11, 12, 13, 14 or 15 min and at most 2 h, 1.5 h, 1 h, 55 min, 50 min, 45 min, 40 min, 39 min, 38 min, 37, min, 36 min, 35 min, 34 min, 33 min, 32 min, 31 min, 30 min, 29 min, 28 min, 27, min, 26 min, 25 min, 24 min, 23 min, 22 min, 21 min, 20 min, 19 min, 18 min, 17, min or 16 min.

The combination of contact time, number of cycles and total process time will be chosen by skilled persons according to the sensitivity of the analyte detection or quantification test they want to implement. This combination will condition the amount of binding partner P1 that will be applied to the inner surface of the solid support.

The temperature at which the application process of the invention is carried out is any temperature compatible with the binding partner P1, i.e. at any temperature that maintains the binding capacities to the analyte of said binding partner P1. In general, the temperature is between 10° C. and 45° C. Room temperature, from about 17° C. to about 25° C., is therefore appropriate. The temperature can also be a function of the instrument used. Thus, with regard to the VIDAS® instrument, the temperature used is 37° C.

Once the binding partner P1 has been applied to the inner surface of the solid support, it can be washed with wash solution W1 to remove unbound partners. The wash solutions useful for this purpose are classic and known to the skilled person. They include buffers, such as phosphate, Tris, HEPES buffers, salts such as NaCl, detergents such as 0.2% Triton X-100, Tween 20, etc. They may also contain saturation agents, also called passivation agents, such as BSA or milk proteins, to saturate the inner surface of the solid support, thus avoiding non-specific bonds that could occur when the test sample and the solid support are contacted. Again, the only condition for this wash solution W1 is that it must not destroy the binding capacity to the analyte of said binding partner P1.

The washing of the inner surface of the solid support may be carried out by any method known to the skilled person, such as soaking the solid support in a container containing said wash solution W1. It can also be implemented by reproducing the suction (ii)/contact (iii)/discharge (iv) cycles as described above. Thus, according to an embodiment of the invention, the application process also includes the following steps, implemented when the suction (ii)/contact (iii)/discharge (iv) cycles are completed:

(v) draw up into the support in which the at least one binding partner P1 is applied, a wash solution W1 contained in a container called container CW1, (vi) continue contact between the wash solution W1 and the inner surface of the solid support for a time between 0 s and 11 min,

(vii) discharge the wash solution W1 into a container, which is optionally said container CW1,

steps (v) to (vii) forming a cycle that can be repeated at least once, over a total duration of at least 1 min and at most 2.5 h.

All the features described above for steps (ii), (iii) and (iv) also apply to steps (v), (vi) and (vii).

As indicated above, at the end of the discharge step, the solution used can be discharged into the wash container CW1 or into another container which can then be the same container as the one used after step (iii). This would then be a waste container which will be identical both for the application of the binding partner P1 and for the wash.

The binding partner P1 can be applied directly to the inner surface of the solid support. Or it can be applied through another binding partner, called binding partner P2 to binding partner P1 to the analyte. Here again, the concepts of analyte and binding partner P2 are quite distinct from each other. The analyte is the compound present in the test sample, while the binding partner P2 is the compound that will bind to the binding partner P1 that will itself bind to the analyte. These binding partners P2 are optionally of immunological origin, as described above. When the binding partner P1 is an antibody, the binding partner P2 can be an anti-species antibody, for example an anti-mouse antibody when the partner P1 is a mouse antibody. The binding partner P2 can also be an anti-IgM antibody when the binding partner P1 is an IgM. When the binding partner P1 is an antigen, for example a peptide, the binding partner P2 can be an anti-peptide antibody. The binding partner P1 may have been previously modified with a ligand, for example a biotin, the binding partner P2 being an antiligand, for example streptavidin. Other examples of binding partners P2 are widely known to the skilled person.

The binding partner P2 can be contained in the sensitization solution S1, which constitutes a particular embodiment of the invention. In this case, the binding partner P2/binding partner P1 formation on the solid support will be implemented during the suction (ii)/contact (iii)/discharge (iv) cycles of the application process of the invention. This is applicable when the partner P2 binds more easily to the support than the partner P1, for example when the partner P2 is streptavidin and the partner P1 is a biotinylated antibody.

The binding partner P2 may also have previously been applied to the inner surface of the solid support according to any process known to the skilled person, for example in static or dynamic mode, reproducing the same cycles as those described above for the application of the binding partner P1.

Thus, at least one binding partner P2 to a binding partner P1 to an analyte to be detected or quantified in a test sample the binding partner P2 can be applied in the tubular solid support, optionally flared, having a circular or ellipsoidal opening at each end, such as a pipette tip or a pipette, by a method comprising the following steps: (a) connect the solid support to a suction-discharge device,

(b) draw up into the solid support, by one of its ends, a solution comprising said at least one binding partner P2, called sensitization solution S2, contained in a container, called container C2,

(c) continue contact between the sensitization solution S2 and the inner surface of the solid support for a time between 0 s and 11 min,

(d) discharge the sensitization solution S2 into a container, which is optionally said container C2,

steps (b) to (d) forming a cycle that can be repeated at least once, over a total duration of at least 1 min and at most 2.5 h.

All the features described above for steps (ii), (iii) and (iv) also apply to steps (b), (c) and (d).

As indicated above, at the end of the discharge step, the solution used can be discharged into the sensitization container C2 or into another container which can then be the same container as the one used with the other discharge steps previously described (waste container).

After application of the binding partner P2 to the inner surface of the solid substrate, it can be washed with a wash solution W2 to remove unbound partners.

Washing may also include the following steps, implemented when the suction (b)/contact (c)/discharge (d) cycles are completed:

(e) draw up into the support in which the at least one binding partner P2 is applied, a wash solution W2 contained in a container called container CW2,

(f) continue contact between the wash solution W2 and the inner surface of the solid substrate for a time of between 0 s and 11 min,

(g) discharge the wash solution W2 into a container, which is optionally said container CW2,

steps (e) to (g) forming a cycle that can be repeated at least once, over a total duration of at least 1 min and at most 2.5 h.

The wash solution W2 has the same features as the wash solution W1 described above. It may be identical (in which case it will be referred to indifferently as wash solution W for solution W1 and solution W2) or it may be different from wash solution W1, both in terms of nature and in terms of its container. It is preferably in a different container CW2 from CW1, but washing can be carried out with the same solution contained in the same container (this is called container CW).

As before, the discharge of the wash solution can be carried out in the wash container itself (CW2 or CW) or in a waste container, for example common for all discharge steps.

Whether or not there are washing steps, in order to use the solid support to detect or quantify an analyte likely to be contained in a test sample, it will then be coated with the binding partner P1. According to an embodiment, the application process of the binding partner P2 also includes the application, in said solid support, after the suction (b)/contact (c)/discharge (d) cycles, of said at least one binding partner P1.

The binding partner P1 can be applied to the inner surface of the solid support according to any process known to the skilled person, for example in static or dynamic mode, such that the same cycles of steps (ii) to (iv) described above can be reproduced for the application of said binding partner P1, which is another embodiment.

The application process of the binding partner P1 can be delayed relative to the application process of the binding partner P2, i.e. there is a waiting time of at least 5 min between the two processes, or it can be implemented immediately after the application process of the binding partner P2. Preferably, they should be used with the same instrument or with two instruments from the same range.

The different containers used in the application process of the binding partners can be physically separated, handled independently from one another, such as vials that are placed in a carousel of an instrument. Or the containers may be integral, for example in the form of a well in a strip, such as the VIDAS® strip (bioMérieux) shown in FIG. 4 (strip 6). According to an embodiment, the container C1, the container CW1, and if necessary the container C2 and/or the container CW2 are contained in the same test strip, which consists of several containers.

Since the test strip or instrument incorporating the different containers is intended for the in vitro detection or quantification of an analyte, it may also include other containers containing other components necessary for the detection or quantification of said analyte. The components necessary for the detection or quantification of said analyte are known to the skilled person and will be described below in the context of the detection or quantification of the analyte. Examples of such components include labelled binding partners, also known as conjugates, wash solutions and detection substrates. Examples of containers include a VIDAS® strip with all reagents (for coating and detection or quantification), or two VIDAS® strips, one for coating and the other for detection or quantification.

The in vitro detection or quantification of an analyte likely to be contained in a test sample may be carried out by any in vitro process known to the skilled person using at least one binding partner P1 to the analyte. It comprises the steps of contacting the test sample with the solid support coated with the binding partner P1 and detecting the binding, if the analyte is present, of said analyte and said at least one binding partner P1.

The application process of the binding partner P1 has been described above. The support can also include a binding partner P2, also applied as described above.

The analyte and the test sample that may contain it are also as described above. According to one embodiment, the test sample is a sample of biological, chemical, food or environmental origin.

The detection or quantification of analyte may be carried out by any analytical method known to the skilled person, such as immunoassay methods. These so-called enzyme-linked immunoassay (EIA) methods are coupled to an enzyme-catalyzed reaction using an enzyme substrate. Depending on the enzyme substrate chosen, a colorimetric signal (enzyme-linked immunosorbent assay, ELISA) (Rassasie, M. J., et al., 1992), a fluorescence signal (enzyme-linked fluorescent assay, ELFA) or a chemiluminescent signal (chemiluminescence immunoassay, CLIA) (Stabler T. V., et al., 1991) can be produced.

These methods are based on measurements to quantify the signals emitted during the analysis of the test sample. The quantity of signals detected is generally proportional to the amount of analyte to be measured (for example in a sandwich test) or inversely proportional to the amount of analyte to be measured (for example in a competition test).

Conventional steps in the in vitro detection and/or quantification process of an analyte by sandwich immunoassay, in a test sample likely to contain said analyte, include or consist of:
the presence of the solid support inside which is applied said at least one binding partner P1 and said sample for fixing the analyte on the partner P1,
the addition of a detection partner, which is directly or indirectly coupled to a label, such as an enzyme capable of lysing an enzymatic substrate, for example fluorogen for ELFA detection, for its binding to the binding partner P1-analyte complex,
when the label is an enzyme, contacting an enzyme substrate and the binding partner P1-analyte-detection partner complex coupled to an enzyme to form a reaction medium, and
detection, for example by immunofluorescence in ELFA detection, of the presence and/or amount of analyte by measuring the signal (for example fluorescence) emitted in the reaction medium.

Detection partner means any partner capable of binding to the analyte to be detected or quantified, which will be coupled directly or indirectly to a label, for example an enzyme. It may be of the same nature as the binding partner P1 or of a different nature. Examples are given above with the binding partner P1.

Direct or indirect coupling of the label to the detection partner means that the label is attached directly to the detection partner recognizing the analyte (direct coupling) or the enzyme is coupled to a binding partner that recognizes the detection partner which recognizes the analyte itself (indirect coupling).

Thus, in the context of direct coupling, the complex formed at the end of the assay, called a conjugate, will consist of: “Capture partner/analyte/detection partner coupled to the label”.

As part of the indirect coupling, the complex formed at the end of the assay will consist of: “Capture partner/analyte/detection partner/binding partner coupled to the label”.

In the latter case, the binding partner is well known to the skilled person and may be, for example, an anti-IgG (immunoglobulin) antibody when the detection partner is an IgG recognizing the analyte of interest.

Label means in particular any molecule containing a reactive group with a group of the detection partner, directly without chemical modification, or after chemical modification to include such a group, which molecule is capable of generating a detectable signal directly or indirectly. A non-exhaustive list of these direct detection markers consists of:

enzymes that produce a detectable signal for example by colorimetry, fluorescence, luminescence, such as horseradish peroxidase, alkaline phosphatase, β-galactosidase, glucose-6-phosphate dehydrogenase,

chromophores such as fluorescent compounds, luminescent compounds, dyes,

radioactive molecules such as 32P, 35S or 125I,

fluorescent molecules such as Alexa or phycocyanins, and

electrochemiluminescent salts such as organometallic derivatives based on acridinium or ruthenium.

According to a particular embodiment, the binding partner P1 used in the detection or quantification process is an immunoassay partner and the detection of the binding of said analyte is implemented by a sandwich test using another binding partner to the analyte, called the detection partner, optionally of a different nature, which is labelled by a label.

Conventional steps in the process of in vitro detection and/or quantification of an analyte by competitive immunoassay in a test sample likely to contain said analyte include:
the presence of the solid support inside which is applied said at least one binding partner P1, an analyte analogue coupled to a label, for example an enzyme capable of lysing an enzymatic substrate, for example fluorogen, and said sample, which compete for binding to the binding partner P1,
when the label is an enzyme, contacting an enzyme substrate, binding partner P1-analyte and binding partner P1-analogue to the analyte complexes to form a reaction medium, and
the detection, for example by immunofluorescence, of the presence and/or amount of analyte by measuring the signal, for example fluorescence, emitted in the reaction medium.

Analogue to the analyte means any molecule that has the same binding capabilities to the binding partner P1 as the analyte.

The label coupled to the analogue to the analyte is equivalent to the label used in a sandwich test.

According to another particular embodiment of the invention, the binding partner P1 used in the detection or quantification process is an immunoassay partner and the detection of the binding or not of said analyte is implemented by a competitive test using a labelled compound competing with the analyte to be detected or quantified, otherwise called the labelled analogue to the analyte.

Regardless of the type of method used, whether in sandwiches or in competition, the enzyme is a widely appropriate label and examples include sulfatase, alkaline phosphatase (ALP), acid phosphatase, glucose oxidase (GOx), glucose-6-phosphate dehydrogenase (G6PD) and β-galactosidase (β-gal). The corresponding enzyme substrates are widely known to the skilled person and include, for example, 4-methylumbelliferyl phosphate or 5-bromo-4-chloro-3-indolyl-beta-D-galactopyranoside.

According to a particular embodiment, in the detection or quantification process according to the invention, the detection of the analyte is carried out using an enzyme and an enzymatic substrate catalyzed by said enzyme, preferably alkaline phosphatase and 4-methylumbelliferyl phosphate.

The analyte detection or quantification process may also include one or more additional washing steps after each step, such as:
before the addition of the detection partner, a washing step to remove the analyte not bound to the binding partner P1-analyte complex; and
after the addition of the detection partner, a washing step to eliminate the unbound detection partner.

The washing steps are steps known to the skilled person. They are implemented with buffers compatible with the reaction medium and signal reading.

The method of detection or quantification of an analyte can be implemented at any time after the application process of the binding partner P1 to the inner surface of the solid support, and if applicable the binding partner P2. It can be implemented one or more days after the application of the binding partner P1 and, if necessary, the binding partner P2, or even one or more weeks later. In this case, the solid support must be dried and then stored in a desiccant bag to avoid any stability problems. Advantageously, the steps of contacting the test sample with the solid support and detecting the binding, if the analyte is present, of said analyte and said at least one binding partner P1 of the detection or quantification process of the invention are carried out immediately after the process of applying at least one binding partner P1 in said solid support as defined above. Immediately after means that the detection or quantification process is implemented within minutes or even seconds of the application process of the binding partner P1. There are no more than 15, 14, 13, 12, 11, 10, 9, 8, 7, 6, 5, 4, 3, 2 or 1 min between the last step of the binding partner P1 application process and the first step of the analyte detection or quantification process. This short time may be useful to introduce the test sample and/or containers that will be used for detection or quantification. In other words, the application process of at least one binding partner P1, or even at least one partner P2 beforehand, and the detection or quantification process are implemented successively.

In order to facilitate the user's organization, to control the costs and space of the laboratory, the analyte detection or quantification process is implemented with the same instrument or type of instrument as that used for the application of the binding partner P1, or even the binding partner P2, or even the different washing processes. Also, the steps of contacting the test sample with the solid support on which said at least one binding partner P1 is applied, and detecting the binding of said analyte and said at least one binding partner P1, include or consist of the following steps consisting in:

(1) drawing up into said solid support, by one of its ends, said sample contained in a container, called container CE, leaving in contact and discharging said sample into a container, which is optionally said container CE,
(2) drawing up into said solid support, at the same end, a solution comprising a compound conjugated to a label, such as another binding partner to the analyte or a compound competing with the analyte, called conjugate solution CS, said conjugate solution C being contained in a container called container CC, leaving in contact and discharging said conjugate solution CS into a container, which is optionally said container CC,
(3) if the label uses a detection substrate, drawing up into said solid support, at the same end, a solution comprising a detection substrate with which the label will react, called substrate solution SS, said substrate solution SS being contained in a container called container CS, leaving in contact and discharging said substrate solution into a container, which is optionally said container CS, and
(4) measuring the transmitted signal.

If necessary, these steps are preceded by a step of connecting the solid support to a suction-discharge device such as a pump unit.

Steps (1) and (2) of suction and discharge can be implemented as described above.

The different components used, for example the binding partners P1, the detection partners, markers, etc. are as described above.

The different containers CE, CC and CS used are as described above and can be separated or contained in the same test strip, for example, which is a particular embodiment.

The last step (4) consists in measuring the transmitted signal. This step is well known to the skilled person. This measurement can be transformed into a representative amount by the user such as the concentration of analyte using a standard curve, also called a calibration curve, the obtaining of which is widely known to the skilled person and can be obtained by i) measuring the signal generated by standards, also called standards or calibrators, then ii) plotting the curve giving the signal as a function of amount or concentration. Very often, it is common practice to find a mathematical model that represents, as accurately as possible, this relationship between signal and amount or concentration, in order to be able to easily calculate the results of a quantitative immunoassay.

In addition to steps (1) to (4), the process may also include washing steps, as described above.

The invention will be better understood using the following examples, which are given by way of non-limiting illustration.

### EXAMPLES

### Example 1: Application of an Anti-TSH Antibody in a Pipette Tip and Detection of TSH

Thyrotropin-releasing hormone or thyroid stimulating hormone (TSH) is a hormone secreted by the thyroid cells of the anterior pituitary gland. This hormone is the main stimulating factor of the thyroid gland that determines the production of the thyroid hormones T3 and T4. In return, these thyroid hormones exert a feedback control on the anterior pituitary gland, which slows down the secretion of TSH. The secretion of TSH is also controlled by the central nervous system through a hypothalamic neuropeptide, TRH, and neurotransmitters such as somatostatin or dopamine. The blood test for TSH is an aid in the diagnosis of thyroid or pituitary disorders.

1.1 Application of an Anti-TSH Antibody in a VIDAS® Pipette Tip According to the Invention

VIDAS® pipette tips are sensitized with 300 μL of a solution of mouse monoclonal anti-TSH antibody (bioMérieux product number 30400) at 2.5 or 5 μg/mL in a Tris HCl buffer pH 7.3 (sensitization solution) contained in one of the wells of a VIDAS® strip, using the VIDAS® instrument, as follows:

### Five-Minute Protocol:

100 μL/s aspiration of the sensitization solution
incubation 10 seconds
solution discharge.

This cycle is repeated 20 times.

### Ten-Minute Protocol:

100 μL/s aspiration of the sensitization solution
incubation 18 seconds
solution discharge.

This cycle is repeated 25 times.

### Fifteen-Minute Protocol:

100 μL/s aspiration of the sensitization solution
incubation 20 seconds
solution discharge.

This cycle is repeated 35 times.

The emptied pipette tips are ready to be used with no further preparation for the determination of TSH in a biological sample.

1.2 Application of an Anti-TSH Antibody in a VIDAS® Pipette Tip According to the Prior Art

For comparison purposes, the VIDAS® pipette tips were sensitized statically as follows: The pipette tips are sensitized with 300 μL of the same mouse anti-TSH monoclonal antibody solution except that the antibody concentration is 6 μg/mL. After about 20 hours at room temperature (18-25° C.) with the sensitization solution, the pipette tips are emptied. Then, 330 μL of a saturation solution containing in particular animal proteins are added for passivation of the pipette tips for about 6 hours. The pipette tips are then emptied, dried and stored at 4° C. until use, away from moisture.

1.3 Determination of TSH in a Sample

The pipette tips coated with anti-TSH antibodies according to points 1.1. and 1.2. above are used with strips from the VIDAS® TSH Kit (bioMérieux product number 30400), which include the other reagents of the immunological reaction.

All assay steps are performed automatically by the VIDAS® instrument according to a standard procedure for this instrument.

The sample contained in the sample well (200 μL) of the strip is collected and transferred to the well containing the alkaline phosphatase (conjugate) labelled anti-TSH antibody. The sample/conjugate mixture is drawn up into and then successively discharged by the pipette tip for about 16 minutes. This operation allows the antigen to bind to the immunoglobulins attached to the pipette tip and to the conjugate forming a “sandwich”.

Three successive washing steps of 3 cycles of 5 seconds each, carried out by the instrument using the wash solution contained in the VIDAS TSH Kit strip, remove the unbound compounds.

In the final detection step, the substrate (4-methylumbelliferyl phosphate) contained in a well of the strip is drawn up into the pipette tip and then discharged into the detection well; the enzyme of the conjugate catalyzes the hydrolysis reaction of this substrate into a product (4-methylumbelliferone) whose emitted fluorescence is measured at 450 nm in the detection well. The value of the fluorescence signal (RFV=relative fluorescence value) is proportional to the concentration of the antigen present in the sample.

Two types of human serum samples containing TSH were used for each concentration condition and protocol duration, namely a sample 1 containing a normal TSH concentration (3 μIU/mL TSH) and a sample 2 containing a high TSH concentration, corresponding to hypothyroidism (40 μIU/mL TSH).

The results are given in FIG. 5 which is a histogram-type representation of the relative fluorescence values (RFVs) for both types of sample 1 and 2 when detecting TSH with the VIDAS® instrument, as a function of the different application conditions of the binding partner (dynamically with a binding partner concentration and contact time that vary—2.5 μg/mL for 5, 10 or 15 min or 5 μg/mL for 5, 10 or 15 min, or static with a concentration at 6 μg/mL for 20 h).

FIG. 5 shows that the RFV signal obtained for sensitization concentrations of 2.5 μg/mL and 5 μg/mL in dynamic mode is quite comparable, regardless of the duration of sensitization. From 5 min of dynamic coating, the RFV signal for sample 1 is about 500 RFV. Sample 2 yields RFVs greater than 3500. All the dynamic application conditions of the anti-TSH antibody used according to the invention have excellent signal dynamics, equivalent to the reference condition obtained in several days by a static incubation process.

### Example 2: Application of a Tg Antigen in a Pipette Tip and Detection of Anti-Tg Antibodies

Thyroglobulin (Tg) is a glycoprotein produced in the thyroid gland and is the main component of follicular colloid. Its main role is the storage and synthesis of thyroid hormones. Antithyroglobulin autoantibodies are often present in patients with autoimmune thyroid disease. For example, they are detected in 30% of patients with Graves' disease (Basedow) and in 85% of patients with Hashimoto's disease (2). Anti-Tg antibodies are associated with hypothyroidism or mild hyperthyroidism and are frequently present in patients with other autoimmune diseases such as rheumatoid arthritis, pernicious anemia and type I diabetes (3, 4).

2.1. Application of a Tg Antigen in a VIDAS® Pipette Tip According to the Invention

VIDAS® pipette tips are sensitized with 300 μL of a native Tg antigen solution (bioMérieux product number 30462) at 7 μg/mL in a phosphate buffer (sensitization solution) contained in one of the wells of a VIDAS® strip, using the VIDAS® instrument, as follows:

### Five-Minute Protocol:

100 μL/s aspiration of the sensitization solution
incubation 10 seconds
solution discharge.

This cycle is repeated 20 times.

### Fifteen-Minute Protocol:

100 μL/s aspiration of the sensitization solution
incubation 20 seconds
solution discharge.

This cycle is repeated 35 times.

### Thirty-Minute Protocol:

100 μL/s aspiration of the sensitization solution
incubation 30 seconds
solution discharge.

This cycle is repeated 50 times.

The emptied pipette tips are ready to be used with no further preparation for the determination of anti-Tg antibodies in a biological sample.

2.2 Application of a Tg Antigen in a VIDAS® Pipette Tip According to the Prior Art

For comparative purposes, the VIDAS® pipette tips were sensitized statically as follows:

The pipette tips are sensitized with 300 μL of the same native Tg antigen solution. After about 6 hours at room temperature (18-25° C.) with the sensitization solution, the pipette tips were emptied. Then, 330 μL of a saturation solution containing in particular animal proteins are added for passivation of the pipette tips for about 6 hours. The pipette tips are then emptied, dried and stored at 4° C. until use, away from moisture.

2.3 Determination of Anti-Tg Antibodies in a Sample

The pipette tips thus coated with Tg antigen according to points 2.1. and 2.2. above are used with strips from the VIDAS anti-TG kit (product number 30462).

The sample (100 μL) is taken by the instrument from the sample well of the strip and then transferred to the well containing a sample diluent. The diluted sample is aspirated and then discharged for about 3 minutes. This step allows the anti-Tg antibodies present in the sample to bind to the antigen attached to the pipette tip. The unbound components of the serum are removed by 3 washes in wells of the VIDAS anti-Tg strip for about 3 minutes. An incubation step with the detection conjugate is performed for about 6 minutes, respecting suction/discharge cycles of 30×8 seconds. The conjugate binds specifically to the anti-Tg antibodies of the previously bound sample. A washing cycle identical to the previous one removes the excess of unbound conjugate before the detection.

In the final detection step, the substrate (4-methylumbelliferyl phosphate) contained in a well of the strip is drawn up into the pipette tip and then discharged into the detection well; the enzyme of the conjugate catalyzes the hydrolysis reaction of this substrate into a product (4-methylumbelliferone) whose emitted fluorescence is measured at 450 nm in the detection well. The value of the fluorescence signal (RFV=relative fluorescence value) is proportional to the concentration of the antigen present in the sample.

The assayed samples are samples of natural human serum with a concentration corresponding to 60 IU/mL (low sample) and 1000 IU/mL (high sample). The blank sample is a mixture of negative samples.

The results are given in FIG. 6 which is a histogram-type representation of the relative fluorescence values (RFVs) for the three sample types (blank, low and high—FIG. 6A) or only for the blank sample (representation of histograms in FIG. 6A with enlarged scale—FIG. 6B) when detecting anti-Tg antibodies with the VIDAS® instrument, as a function of the different application conditions of the binding partner (dynamically for 5, 15 or 30 minutes, or statically for about 6 hours).

FIG. 6A shows that the dynamic application conditions for binding the Tg antigen to the support according to the process of the invention give signals equivalent to or better than those obtained with the static reference condition. The signal obtained on low and high human serum samples is satisfactory after 5 minutes of dynamic adsorption time. It is at its maximum for the 30-minute protocol, allowing a higher signal than the reference with the high sample.

The signal of the blank sample is used to evaluate the non-specific signal. The signal for this sample must be at the lowest level. As shown in FIG. 6B, with dynamic application conditions, this non-specific signal is weaker (less than 20 RFV) than for the static reference for which the signal is 25 RFV.

The conditions of application of the Tg antigen on the support according to the invention lead to an immunoassay equivalent to or better than the reference condition.

### Example 3: Application of Several Binding Partners to Form a Biotinylated BSA/Streptavidin/Biotinylated Anti-cTni Antibody Complex and Detection of TNI

Troponin is a protein complex that sensitizes muscle cells to the calcium responsible for inhibiting the binding between myosin and actin (by masking the actin site that is used for binding to myosin). It therefore has an inhibitory function that has the effect of initiating muscle relaxation. protein used. Troponin I (TnI) is a subunit. Its determination is widely used as a tool to assist in the diagnosis of myocardial infarction (MI) and 30-day risk stratification for all-cause mortality and major adverse cardiac events (MACE) including myocardial infarction and revascularization in patients with acute coronary syndrome (ACS)-like symptoms.

3.1 Dynamic Sensitization of Pipette Tips with Biotinylated BSA and Streptavidin for the Binding of Specific Biotinylated Anti-cTni Antibodies

VIDAS® pipette tips are sensitized with 300 μL of a biotinylated BSA solution (bioMérieux product number 30448) at 1 μg/mL in a carbonate buffer contained in one of the wells of a VIDAS® strip. The cycles performed by the VIDAS® and repeated 50 times are:
100 μL/s aspiration of the biotinylated BSA solution,
incubation for 20 s then
solution discharge.

Next, these same pipette tips are incubated with 300 μL of a 5 μg/mL streptavidin solution (bioMérieux product number 30448) diluted in PBS buffer for 50 cycles of 20 seconds (aspiration, incubation of 20 s and discharge).

The pipette tips are then sensitized with a mixture of biotinylated anti-cTnI antibodies (bioMérieux product number 30448) at 2 μg/mL and 3 μg/mL respectively, on 300 μL. The incubation time is 50 times 20 seconds.

After each of these 3 incubation steps, the pipette tips are washed twice successively on 300 μL for 8 times 1 second in wells containing a wash solution in the VIDAS cartridge. The emptied pipette tips are ready to be used with no further preparation for the determination of TnI in a biological sample.

3.2. Application of Biotinylated BSA and then Streptavidin for the Binding of Specific Biotinylated Anti-cTni Antibodies in a VIDAS® Pipette Tip According to the Prior Art

For comparative purposes, the VIDAS® pipette tips were sensitized statically as follows:

The pipette tips are sensitized with 300 μL of the same biotinylated BSA solution for about 20 hours at room temperature (18-25° C.). The pipette tips are then emptied and filled with 300 μL of streptavidin solution for about 20 hours at room temperature (18-25° C.). After emptying the pipette tips, the sensitization step on approximately 300 with the mixture of biotinylated anti-cTni antibodies at the same concentration as in point 3.1. continues for approximately 20 hours. The pipette tips are then emptied, dried and stored at 4° C. until use, away from moisture.

3.2. Determination of cTnI

All test steps are performed automatically by the instrument according to a standard instrument operating mode. They consist of a succession of suction/discharge cycles of the reaction medium. The sample (200 μL) is collected and transferred to the well containing the alkaline phosphatase (conjugate) labelled cardiac anti-troponin antibodies. The sample/conjugate mixture is drawn up into and then successively discharged by the pipette tip for about 10 minutes (50×8 seconds). This operation allows the antigen to bind to the immunoglobulins attached to the pipette tip and to the conjugate forming a “sandwich”.

Three successive washing steps of 3 cycles of 2 seconds each remove unbound compounds.

Two detection steps are then carried out successively. At each step, the substrate (4-methylumbelliferyl phosphate) is drawn up into and then discharged into the pipette tip; the enzyme of the conjugate catalyzes the hydrolysis reaction of this substrate into a product (4-methylumbelliferone) whose emitted fluorescence is measured at 450 nm. The value of the fluorescence signal is proportional to the concentration of the antigen present in the sample.

At the end of the test, the results are automatically calculated by the instrument against two stored calibration curves corresponding to the two detection steps. A threshold signal controls the choice of the calibration curve to be used for each sample. Then the results are printed.

The assayed samples are human sera with Tni concentrations ranging from 0.001 μg/L to 14.03 μg/L (samples 1 to 11).

RFV signal results as a function of TnI concentration are given in FIG. 7 (FIG. 7A: samples 1 to 6 and FIG. 7B: samples 7 to 11) which shows that the signal is equivalent between the two types of coating, regardless of the concentration of the sample tested. The application of the biotinylated BSA/streptavidin/biotinylated antibody complex according to the invention makes it possible to obtain an efficient immunoassay, according to a much faster process than with the prior art.

### Example 4: Variation of Binding Partner Concentrations in the Sensitization Solution

4.1. Application of an Anti-TSH Antibody in a VIDAS® Pipette Tip According to the Invention

VIDAS® pipette tips are sensitized with 300 μL of a 4 μg/mL solution of mouse monoclonal anti-TSH antibody in a Tris HCl buffer pH 7.3 (sensitization solution) contained in one of the wells of a VIDAS® strip, using the VIDAS® instrument, as follows:

### Sixty-Minute Protocol:

100 μL/s aspiration of the sensitization solution
incubation 20 seconds
solution discharge.

This cycle is repeated 139 times.

The empty pipette tips are ready for use with no further preparation for the determination of TSH in biological samples.

4.2. Application of an Anti-TSH Antibody in a VIDAS® Pipette Tip According to the Prior Art

For comparative purposes, the VIDAS® pipette tips are sensitized according to the prior art as follows:

The pipette tips are sensitized with 300 μL of a solution of mouse monoclonal anti-TSH antibody (bioMérieux product number 30400) at 6 μg/mL in a Tris HCl pH 7.3 buffer. After about 20 hours at room temperature (18-25° C.) with the sensitization solution, the pipette tips are emptied. Then, 330 μL of the same solution containing animal proteins are added for passivation of the pipette tips for about 6 hours. The pipette tips are then emptied, dried and stored at 4° C. until use, away from moisture.

4.3. Determination of TSH

The TSH assay is implemented as described in Example 1. The assayed samples (SC13, SC14, SC15, SC16 and SC17) are human sera with increasing TSH concentrations.

The results of these assays are given in FIG. 8 which is a histogram-type representation giving the RFV signal for each sample using either a pipette tip coated with an anti-TSH antibody according to the invention (dynamic coating at 4 μg/mL) or a pipette tip coated with an anti-TSH antibody according to the prior art (static coating at 6 μg/mL).

FIG. 8 shows that the signals obtained between the two pipette tip fabrications (according to the invention and according to the prior art) are very comparable, whereas the fabrication of the invention uses ⅓ less anti-TSH antibodies in the sensitization solution (4 μg/mL versus 6 μg/mL) and allows a production of these pipette tips for use in immunoassay in 1 hour versus 2 steps of more than 6 hours each according to the prior art. All other experimental conditions being identical, the application of anti-TSH antibodies according to the invention allows substantial savings in raw materials and time.

### REFERENCES

Boersma Y L and Plütckthun A, 2011, Curr. Opin. Biotechnol, 22:849-857
Ellington A D and Szostak J W., 1990, Nature, 346:818-822
Rassasie M. J. et al., 1992, Steroids, 57:112
Stabler T. V., et al., 1991, Clin. Chem., 37(11):1987

1. A process for applying, in a tubular solid support, optionally flared, having a circular or ellipsoidal opening at each end, at least one binding partner P1 to an analyte to be detected or quantified in a test sample, comprising the following steps:
(i) connect the solid support to a suction-discharge device,
(ii) draw up into the support, by one of its ends, a solution comprising the at least one binding partner P1, called sensitization solution S1, contained in a container, called container C1,
(iii) continue contact between the sensitization solution S1 and the inner surface of the solid support for a time between 0 s and 11 min,
(iv) discharge the sensitization solution S1 into a container, which is optionally the container C1,
steps (ii) to (iv) forming a cycle that can be repeated at least once, over a total duration of at least 1 min and at most 2.5 h.

2. The application process as claimed in claim 1, wherein the contact time of step (iii) between the sensitization solution S1 and the inner surface of the solid support is between 2 s and 1 min.

3. The application process as claimed in claim 1, wherein steps (ii) to (iv) are repeated from 10 to 100 times.

4. The application process as claimed in claim 1, wherein the total of the repeated cycles is between 10 and 20 min.

5. The application process as claimed in claim 1, wherein it also includes the following steps, carried out when the suction (ii)/contact (iii)/discharge (iv) cycles are completed:
(v) draw up into the solid support in which the at least one binding partner P1 is applied, a wash solution W1 contained in a container called container CW1,
(vi) continue contact between the wash solution W1 and the inner surface of the solid support for a time between 0 s and 11 min,
(vii) discharge the wash solution W1 into a container, which is optionally the container CW1,
steps (v) to (vii) forming a cycle that can be repeated at least once, over a total duration of at least 1 min and at most 2.5 h.

6. The application process as claimed in claim 1, wherein the sensitization solution S1 also contains at least one binding partner P2 to the binding partner P1 to the analyte.

7. A process for applying, in a tubular solid support, optionally flared, having a circular or ellipsoidal opening at each end, at least one binding partner P2 to a binding partner P1 to an analyte to be detected or quantified in a test sample, comprising the following steps:
(a) connect the solid support to a suction-discharge device,
(b) draw up into the solid support, by one of its ends, a solution comprising the at least one binding partner P2, called sensitization solution S2, contained in a container, called container C2,
(c) continue contact between the sensitization solution S2 and the inner surface of the solid support for a time between 0 s and 11 min,
(d) discharge the sensitization solution S2 into a container, which is optionally the container C2,
steps (b) to (d) forming a cycle that can be repeated at least once, over a total duration of at least 1 min and at most 2.5 h.

8. The application process as claimed in claim 7, wherein it also includes the following steps, carried out when the suction (b)/contact (c)/discharge (d) cycles are completed:
(e) draw up into the support in which the at least one binding partner P2 is applied, a wash solution W2 contained in a container called container CW2,
(f) continue contact between the wash solution W2 and the inner surface of the solid substrate for a time between 0 s and 11 min,
(g) discharge the wash solution W2 into a container, which is optionally the container CW2,
steps (e) to (g) forming a cycle that can be repeated at least once, over a total duration of at least 1 min and at most 2.5 h.

9. The application process as claimed in claim 7, also comprising the application, in the solid support, after the suction (b)/contact (c)/discharge (d) cycles, of the at least one binding partner P1.

10. (canceled)

11. The application process as claimed in claim 5, wherein the container C1, the container CW1, and optionally the container C2 and/or the container CW2 are contained in the same test strip, which consists of several containers.

12. The application process as claimed in claim 11, wherein the test strip also includes other containers containing other components necessary for the detection or quantification of the analyte.

13. A process for the in vitro detection or quantification of an analyte in a test sample likely to contain the analyte, the process using at least one tubular solid support, optionally flared, having a circular or ellipsoidal opening at each end, in which at least one binding partner P1 to an analyte to be detected or quantified is applied in the test sample according to an application process as defined in claim 1, which detection or quantification process comprises the steps of contacting the test sample with the solid support and detecting the binding, if the analyte is present, of the analyte and the at least one binding partner P1.

14. The detection or quantification process as claimed in claim 13, wherein the binding partner P1 is an immunoassay partner and the detection of the binding of the analyte is carried out by a sandwich test using another binding partner to the analyte, optionally of a different nature, which is labelled.

15. The detection or quantification process as claimed in claim 13, wherein the binding partner P1 to the analyte is an immunoassay partner and the detection or not of the binding of the analyte is carried out by a competitive test using a labelled compound competing with the analyte to be detected or quantified.

16. The detection or quantification process as claimed in claim 13, wherein the detection of the analyte is carried out using an enzyme and an enzymatic substrate catalyzed by the enzyme.

17. (canceled)

18. The detection or quantification process as claimed in claim 13, wherein the steps of contacting the test sample with the solid support on which the at least one binding partner P1 is applied, and detecting the binding of the analyte and the at least one binding partner P1, include the following steps consisting in:
(1) drawing up into said solid support, by one of its ends, the sample contained in a container, called container CE, leaving it in contact and discharging the sample into a container, which is optionally the container CE,
(2) drawing up into the solid support, at the same end, a solution comprising a compound conjugated to a label, called conjugate solution CS, the conjugate solution C being contained in a container called container CC, allowing the conjugate solution CS to contact and discharge into a container, which is optionally the container CC,
(3) if the label uses a detection substrate, drawing up into the solid support, at the same end, a solution comprising a detection substrate with which the label will react, called substrate solution SS, the substrate solution SS being contained in a container called container CS, leaving in contact and discharging the substrate solution into a container, which is optionally the container CS, and
(4) measuring the transmitted signal.

19. The detection or quantification process as claimed in claim 18, wherein the container CE, the container CC and the container CS are contained in the same test strip.

20. The detection or quantification process as claimed in claim 13, wherein the test sample to be analyzed is a sample of biological, food or environmental origin.
